(19) United States
(12) Patent Application Publication
(10) Pub. No.: US 2014/0296348 A1
(43) Pub. Date: Oct. 2, 2014

(54) OPHTHALMOLOGICAL AQUEOUS COMPOSITION

(71) Applicant: ROHTO PHARMACEUTICAL CO., LTD., Osaka-shi, Osaka (JP)

(72) Inventors: Yasuko Matsumura, Osaka-shi (JP); Chinatsu Furumiya, Osaka-shi (JP); Masashi Itoh, Osaka-shi (JP)

(21) Appl. No.: 14/355,383

(22) PCT Filed: Oct. 31, 2012
(86) PCT No.: PCT/JP2012/078126

§ 371 Date: Apr. 30, 2014

(30) Foreign Application Priority Data
Nov. 1, 2011 (JP) 2011-240145

(51) Int. Cl. A61K 47/02; A61K 31/045

(52) U.S. Cl. CPC A61K 47/02; A61K 31/045
USPC 514/690; 514/724; 514/692; 514/729; 514/739

(57) ABSTRACT

The present invention provides an aqueous ophthalmic composition having a pH of 7 or more, containing a terpenoid and zinc chloride. According to the present invention, an aqueous ophthalmic composition is provided in which adsorption of the terpenoid to a container can be inhibited, thereby making it possible to inhibit the reduction in the terpenoid content for a long period of time, the aqueous ophthalmic composition having some excellent actions such as an inhibitory action for histamine release, and an inhibitory action for discharges from the eyes.

### TECHNICAL FIELD

The present invention relates to an aqueous ophthalmic composition. More particularly, the present invention relates to an aqueous ophthalmic solution in which adsorption of a terpenoid to a container is inhibited, and a method for inhibiting adsorption of a terpenoid to a container in an aqueous ophthalmic composition.

### BACKGROUND ART

In ophthalmic compositions, a terpenoid such as menthol is contained in order to give a refreshing feel. However, when an ophthalmic composition containing a terpenoid is filled in a plastic container or the like, the terpenoid adsorbs to the container during storage, so that the content of the terpenoid is reduced. The reduction in the content of the terpenoid has some disadvantages of impairing feel of use because the reduction greatly influences the senses of the ophthalmic compositions, and further impairing qualities of the ophthalmic compositions. On the other hand, a method of inhibiting adsorption of a terpenoid to a plastic container by adding a surfactant (Patent Publications 1 and 2). However, surfactants may irritate ocular mucous membranes, so that it is said that some side effects are caused, such as disorders are caused in the cornea if, for example, eye drops containing surfactants are frequently dropped or if individuals having disorders in the cornea or individuals whose tear flow is not normal showing dry eye symptoms and the like are dropped, so that there are some concerns in the aspect of safety.

On the other hand, a zinc salt such as zinc sulfate or zinc lactate has constrictive actions and anti-inflammatory actions, and has been used widely in eye drops as constrictive agents and anti-inflammatory agents, and zinc chloride or zinc sulfate has also been known as a bactericidal agent. However, the influences which these components would have on the aqueous ophthalmic compositions containing terpenoids have not been elucidated.

### PRIOR ART REFERENCES

### Patent Publications

Patent Publication 1: Japanese Patent Laid-Open No. 2002-003364
Patent Publication 2: Japanese Patent Laid-Open No. 2005-162747

### SUMMARY OF THE INVENTION

### Problems to be Solved by the Invention

The present invention has been accomplished in view of the current situations of the prior art mentioned above, and an object thereof is to provide an aqueous ophthalmic composition containing a terpenoid, the aqueous ophthalmic composition capable of inhibiting the adsorption of terpenoids to the container, thereby maintaining a high residual ratio of terpenoids in the aqueous ophthalmic composition, and further to provide a method for inhibiting the adsorption of the terpenoids contained in the aqueous ophthalmic composition.

Furthermore, another object of the present invention is to provide an aqueous ophthalmic composition having other more improved actions.

### Means to Solve the Problems

The present inventor has made intensive studies in order to accomplish the objects mentioned above. As a result, it has been found that in the aqueous ophthalmic composition having a pH of 7 or more, containing zinc chloride together with a terpenoid, the adsorption of the terpenoid to the container can be inhibited when the aqueous ophthalmic composition is filled in various containers such as plastic containers and stored, thereby making it possible to inhibit the reduction in the terpenoid content for a long period of time. In addition, the present inventor has found that the aqueous ophthalmic composition containing the above ingredients markedly has an inhibitory action for histamine release, and further has an unexpected action of effectively inhibiting discharges from the eyes. The present invention has been perfected as a result of further studies based on these findings.

In other words, the present invention provides an aqueous ophthalmic composition of the embodiments listed hereinbelow.

Item 1-1: An aqueous ophthalmic composition having a pH of 7 or more, containing a terpenoid and zinc chloride.
Item 1-2: The aqueous ophthalmic composition according to the above item 1-1, wherein the terpenoid is at least one member selected from the group consisting of menthol, menthone, camphor, borneol, and geraniol.
Item 1-3. The aqueous ophthalmic composition according to the item 1-1 or 1-2, wherein the content proportion of the terpenoid in total is from 0.00001 to 0.2 w/v %, on the basis of a total amount of the aqueous ophthalmic composition.
Item 1-4: The aqueous ophthalmic composition according to any one of the above items 1-1 to 1-3, wherein the zinc chloride is contained in an amount of from 0.000005 to 5,000 parts by weight, based on 1 part by weight of a total amount of the terpenoids.
Item 1-5: The aqueous ophthalmic composition according to any one of the above items 1-1 to 1-4, further comprising a surfactant.
Item 1-6: The aqueous ophthalmic composition according to any one of the above items 1-1 to 1-5, further comprising a cellulose-based polymeric compound.
Item 1-7: The aqueous ophthalmic composition according to any one of the above items 1-1 to 1-6, wherein the composition is housed in a container made of a material containing at least one plastic selected from the group consisting of polyethylene terephthalate resins, polypropylene resins, polyethylene resins, and polyethylene naphthalate resins.

Also, the present invention provides a method for inhibiting adsorption of a terpenoid to a container in the aqueous ophthalmic composition, or a method for inhibiting the reduction in the content of a terpenoid, of the embodiments listed hereinbelow.

Item 2-1: A method for inhibiting adsorption of a terpenoid to a container in an aqueous ophthalmic composition having a pH of 7 or more, including combining in the aqueous ophthalmic composition zinc chloride together with a terpenoid.
Item 2-2: A method for inhibiting the reduction in the content of a terpenoid in an aqueous ophthalmic composition having a pH of 7 or more, including combining in the aqueous ophthalmic composition zinc chloride together with a terpenoid.

Further, the present invention also provides a method for enhancing an inhibitory action for histamine release of the aqueous ophthalmic composition, or a method for giving an inhibitory action for discharges from the eyes to the aqueous ophthalmic composition, of the embodiments listed hereinbelow.

Item 3-1. A method for enhancing an inhibitory action for histamine release of an aqueous ophthalmic composition having a pH of 7 or more, including combining in the aqueous ophthalmic composition a terpenoid and zinc chloride.
Item 3-2. A method for giving an inhibitory action for discharges from the eyes to the aqueous ophthalmic composition having a pH of 7 or more, including combining in the aqueous ophthalmic composition a terpenoid and zinc chloride.

Further, the present invention also provides a method for inhibiting or treating itchiness of the eyes, or a method for inhibiting discharges from the eyes, of the embodiments listed hereinbelow.

Item 4-1. A method for inhibiting or treating itchiness of the eyes, including contacting an aqueous ophthalmic composition having a pH of 7 or more, containing a terpenoid and zinc chloride, with cornea and/or conjunctiva.
Item 4-2. A method for inhibiting discharges from the eyes, including contacting an aqueous ophthalmic composition having a pH of 7 or more, containing a terpenoid and zinc chloride, with cornea and/or conjunctiva.

Further, the present invention also provides use of the embodiments listed hereinbelow.

Item 5. Use of a terpenoid and zinc chloride, in the manufacture of an aqueous ophthalmic composition having a pH of 7 or more, containing a terpenoid and zinc chloride, the aqueous composition having an action for inhibiting adsorption to a container of a terpenoid, an enhanced inhibitory action for histamine release, or an inhibitory action for discharges from the eyes.

Further, the present invention also provides use of the embodiments listed hereinbelow.

Item 6-1. Use of a composition having a pH of 7 or more, containing a terpenoid and zinc chloride, as an aqueous ophthalmic composition having an action for inhibiting adsorption to a container of a terpenoid, an enhanced inhibitory action for histamine release, or an inhibitory action for discharges from the eyes.
Item 6-2. Use according to the above item 6-1, wherein the composition is a composition as defined in any one of the above items 1-2 to 1-7.

Further, the present invention also provides a composition of the embodiments listed hereinbelow.

Item 7-1. An aqueous composition having a pH of 7 or more, containing a terpenoid and zinc chloride, for use in an aqueous ophthalmic composition having an action for inhibiting adsorption to a container of a terpenoid, an enhanced inhibitory action for histamine release, or an inhibitory action for discharges from the eyes.
Item 7-2. The composition according to the above item 7-1, wherein the composition is a composition as defined in any one of the above items 1-2 to 1-7.

Further, the present invention also provides a method for producing an aqueous ophthalmic composition of the embodiments listed hereinbelow.

Item 8-1. A method for producing an aqueous ophthalmic composition having an action for inhibiting adsorption to a container of a terpenoid, an enhanced inhibitory action for histamine release, or an inhibitory action for discharges from the eyes, including adding a terpenoid and zinc chloride to a carrier containing water, to provide an aqueous composition having a pH of 7 or more.
Item 8-2. The method according to the above item 8-1, wherein the composition is a composition as defined in any one of the above items 1-2 to 1-7.

### Effects of the Invention

According to the aqueous ophthalmic composition of the present invention, in an aqueous ophthalmic composition containing a terpenoid, the adsorption of the terpenoid to the container is inhibited, so that the reduction in the content of the terpenoid can be inhibited in the aqueous ophthalmic composition over a long period of time even during, for example, distribution process and the like. Since the reduction in the terpenoid content in the aqueous ophthalmic composition greatly affects feel of use, the compliance of patients can also be improved by inhibiting the reduction in the terpenoid content.

Further, the aqueous ophthalmic composition of the present invention has an excellent action of inhibiting histamine release. Accordingly, the composition of the present invention is used as an eye drop or an eyewash to contact the composition with cornea by a method such as dropping or eye-washing, thereby anti-histamine action is enhanced, so that the itchiness of the eyes can be inhibited or treated. Therefore, the aqueous ophthalmic composition of the present invention is useful as an eye drop or eyewash for inhibiting itchiness, and the composition is further useful as eye drops or the like for allergies, for inflammation, or for dry eyes, for wearing or putting on contact lens that accompany itchy symptoms.

In addition, the aqueous ophthalmic composition of the present invention has an action of effectively inhibiting discharges from the eyes. Accordingly, the composition of the present invention is contacted with cornea by a method such as dropping or eye-washing, whereby the amount of discharges from the eyes can be inhibited against patients showing symptoms of discharges from the eyes, so that, for example, ease in opening eyes, ease in blinking, blurriness of eyes, or the like can be ameliorated.

### MODES FOR CARRYING OUT THE INVENTION

### 1. Aqueous Ophthalmic Composition

The aqueous ophthalmic composition of the present invention is an aqueous composition having a pH of 7 or more, containing a terpenoid and zinc chloride.

The term “aqueous composition” as used herein is a composition containing water. The content proportion of water in the aqueous ophthalmic composition of the present invention is, for example, from 10 to 99.8 w/v %, preferably from 55 to 99.0 w/v %, more preferably from 70 to 98.0 w/v %, even more preferably from 85 to 98.0 w/v %, and especially preferably from 90 to 98.0 w/v %, on the basis of a total amount of the hydrophobic ophthalmic composition.

The aqueous ophthalmic composition of the present invention will be explained concretely hereinbelow.

(1) Terpenoid

The terpenoid is a known compound having a structure having an isoprene unit as a constituting unit, which has been used as a cooling agent.

In the aqueous ophthalmic composition of the present invention, the terpenoid can be used without particular limitations so long as the terpenoid is pharmacologically (pharmaceutically) or physiologically acceptable in the field of medicine. The terpenoid as described above concretely includes menthol, menthone, camphor, borneol, geraniol, cineol, citronellol, carvone, anethole, eugenol, limonene, linalool, linalyl acetate, derivatives thereof, and the like. These compounds may be in any of d-form, l-form, and dl-form.

In addition, in the present invention, an essential oil containing the above compound may be used as a terpenoid. The essential oil as mentioned above includes, for example, eucalyptus oil, bergamot oil, peppermint oil, cool-mint oil, spearmint oil, Japanese mint oil, fennel oil, cinnamon oil, rose oil, camphor oil, and the like. These terpenoids may be used alone, or two or more kinds may be optionally combined and used.

Among these terpenoids, menthol, menthone, camphor, borneol, geraniol, and the like are preferred, menthol and camphor are more preferred, l-menthol, dl-menthol, d-camphor, and dl-camphor are even more preferred, and l-menthol is especially preferred.

The content proportion of the terpenoid in the aqueous ophthalmic composition of the present invention can be appropriately set depending upon the concrete kinds of the ophthalmic composition. As one example, the content proportion of the terpenoid is in total from 0.00001 to 0.5 w/v %, preferably from 0.0005 to 0.25 w/v %, and more preferably from 0.001 to 0.1 w/v %, on the basis of a total amount of the aqueous ophthalmic composition. In addition, it is needless to say that the content proportion can be increased or decreased, by the number of administration, the method of administration, or the like.

(2) Zinc Chloride

In the aqueous ophthalmic composition of the present invention, zinc chloride is combined together with a terpenoid, thereby inhibiting adsorption of a terpenoid to a plastic container, so that the reduction in the content of the terpenoid can be inhibited in the aqueous ophthalmic composition over a long period of time. Further, the aqueous ophthalmic composition of the present invention has an excellent action such as an action for inhibiting histamine release or an action for inhibiting discharges from the eyes, so that some effects such as enhancement of anti-histamine action and inhibition of the amount of discharges from the eyes are exhibited by the use of the aqueous ophthalmic composition.

Zinc chloride can be used without particular limitations, so long as zinc chloride can be used in the aqueous ophthalmic composition. For example, zinc chloride as prescribed in The Japanese Pharmacopeia Sixteenth Edition can be used.

The content proportion of the zinc chloride in the aqueous ophthalmic composition of the present invention is not particularly limited. As one example, the content proportion of the zinc chloride is from 0.000001 to 0.05 w/v %, preferably from 0.00005 to 0.025 w/v %, and more preferably from 0.0001 to 0.015 w/v %, on the basis of a total amount of the aqueous ophthalmic composition.

In addition, the content proportion of zinc chloride based on the content of the terpenoid contained in the aqueous ophthalmic composition is not particularly limited, and, for example, zinc chloride is in an amount of from 0.000005 to 5,000 parts by weight, preferably from 0.0005 to 1,000 parts by weight, more preferably from 0.002 to 500 parts by weight, especially preferably from 0.002 to 10 parts by weight, and most preferably from 0.002 to 1 part by weight, based on 1 part by weight of a total amount of the terpenoid, contained in the aqueous ophthalmic composition.

(3) pH of Aqueous Ophthalmic Composition

In the aqueous ophthalmic composition of the present invention, when a pH value of the aqueous ophthalmic composition is 7 or more, adsorption of the terpenoid to a container can be inhibited, and further gives actions such as enhancement of inhibitory effects for histamine release and inhibition of discharges from the eyes, by combining zinc chloride together with the terpenoid. The concrete pH value is varied depending on intended applications, usage form, and the like, and the pH is, for example, from 7 to 9.5, preferably from 7 to 9, and more preferably from 7 to 8.5.

In the aqueous ophthalmic composition of the present invention, an adjustment of a pH can be performed using the above pH adjustment agent, buffer or the like.

(4) Surfactant

The aqueous ophthalmic composition of the present invention contains a terpenoid and zinc chloride, and the aqueous ophthalmic composition can optionally contain a surfactant. Since the aqueous ophthalmic composition contains a surfactant, the effects of the present invention, in other words, the effects such as inhibition of adsorption of the terpenoid to the container, enhancement of inhibitory action for histamine release, and inhibition of discharges from the eyes are more remarkably exhibited.

The surfactant which can be contained in the aqueous ophthalmic composition of the present invention is not particularly limited, with the limit of being pharmacologically (pharmaceutically) or physiologically acceptable in the field of medicine, and the surfactant may be any one of nonionic surfactants, amphoteric surfactants, anionic surfactants, and cationic surfactants. Among them, concrete examples of the nonionic surfactants include POE sorbitan fatty acid esters such as POE(20) sorbitan monolaurate (Polysorbate 20), POE(20) sorbitan monopalmitate (Polysorbate 40), POE(20) sorbitan monostearate (Polysorbate 60), POE(20) sorbitan tristearate (Polysorbate 65), and POE(20) sorbitan monooleate (Polysorbate 80); POE-POP block copolymers such as Poloxamer 407, Poloxamer 235, Poloxamer 188, Poloxamer 403, Poloxamer 237, and Poloxamer 124; POE hydrogenated castor oils such as POE(60) hydrogenated castor oil (polyoxyethylene hydrogenated castor oil 60); POE alkyl ethers such as POE(9) lauryl ether; POE-POP alkyl ethers such as POE(20)POP(4) cetyl ether; POE alkyl phenyl ethers such as POE(10) nonyl phenyl ethers; and the like. Here, in the compounds exemplified above, POE stands for polyoxyethylene, POP stands for polyoxypropylene, and the numbers inside parentheses stand for the number of moles added. In addition, the amphoteric surfactants which can be contained in the aqueous ophthalmic composition of the present invention are concretely exemplified by alkyldiaminoethyl glycines, and the like. In addition, the cationic surfactants which can be contained in the aqueous ophthalmic composition of the present invention are concretely exemplified by benzalkonium chloride, benzethonium chloride, and the like. Also, the anionic surfactants which can be contained in the aqueous ophthalmic composition of the present invention are concretely exemplified by alkylbenzenesulfonates, alkyl sulfates, polyoxyethylene alkyl sulfates, α-sulfomethyl esters of fatty acids, α-olefinsulfonates, and the like.

In the aqueous ophthalmic composition of the present invention, the surfactants may be used in a single kind alone or in a combination of two or more kinds.

The content proportion of the surfactant in the aqueous ophthalmic composition of the present invention is not particularly limited, and as one embodiment, the content proportion of the surfactant in a total amount is preferably from 0.001 to 5 w/v %, more preferably from 0.01 to 1 w/v %, and even more preferably from 0.03 to 0.5 w/v %, on the basis of a total amount of the aqueous ophthalmic composition.

More specifically, the following content proportions are exemplified.

In a case where the surfactant is a nonionic surfactant, the content proportion of the nonionic surfactant in a total amount is preferably from 0.001 to 2 w/v %, more preferably from 0.01 to 1 w/v %, and even more preferably from 0.03 to 0.5 w/v %, on the basis of a total amount of the aqueous ophthalmic composition.

In a case where the surfactant is a amphoteric surfactant, the content proportion of the amphoteric surfactant in a total amount is preferably from 0.001 to 1 w/v %, more preferably from 0.005 to 0.5 w/v %, and even more preferably from 0.01 to 0.1 w/v %, on the basis of a total amount of the aqueous ophthalmic composition.

In a case where the surfactant is an anionic surfactant, the content proportion of the anionic surfactant in a total amount is preferably from 0.001 to 2 w/v %, more preferably from 0.01 to 1 w/v %, and even more preferably from 0.03 to 0.5 w/v %, on the basis of a total amount of the aqueous ophthalmic composition.

In a case where the surfactant is a cationic surfactant, the content proportion of the cationic surfactant in a total amount is preferably from 0.001 to 1 w/v %, more preferably from 0.005 to 0.5 w/v %, and even more preferably from 0.01 to 0.1 w/v %, on the basis of a total amount of the aqueous ophthalmic composition.

(5) Polymeric Compound

Also, the aqueous ophthalmic composition of the present invention can optionally contain a polymeric compound. Since the aqueous ophthalmic composition contains a polymeric compound, the effects of the present invention, in other words, the effects such as inhibition of adsorption of the terpenoid to the container, enhancement of inhibitory action for histamine release, and inhibition of discharges from the eyes are more remarkably exhibited. The polymeric compounds may be used in a single kind alone or in a combination of two or more kinds.

As a polymeric compound, for example, a cellulose-based polymeric compound can be used. As the cellulose-based polymeric compound, cellulose, a cellulose derivative in which a hydroxy group of the cellulose is substituted with another functional group, a salt thereof, or the like can be used.

Concrete examples of the cellulose derivative include methyl cellulose, ethyl cellulose, hydroxyethyl cellulose, hydroxymethyl cellulose, hydroxypropyl cellulose, hydroxypropylmethyl cellulose (hypromellose), carboxymethyl cellulose, carboxymethyl cellulose sodium, carboxyethyl cellulose, and the like.

The salts of the cellulose and derivatives thereof are not particularly limited, so long as the salts are pharmacologically (pharmaceutically) or physiologically acceptable in the field of medicine. The salts can be exemplified by alkali metal salts such as sodium salts and potassium salts.

Among the cellulose-based polymeric compounds, hydroxypropylmethyl cellulose, hydroxyethyl cellulose, carboxymethyl cellulose, and carboxymethyl cellulose sodium are preferred, and hydroxypropylmethyl cellulose and hydroxyethyl cellulose are more preferred.

These cellulose-based polymeric compounds may be used as a single kind alone, or in a combination of two or more kinds.

Here, the content proportion of the cellulose-based polymeric compound is such that a total amount of the cellulose-based polymeric compounds is from 0.0001 to 10 w/v %, preferably from 0.0025 to 7 w/v %, more preferably from 0.005 to 5 w/v %, especially preferably from 0.01 to 3 w/v %, and most preferably from 0.05 to 2.5 w/v % or so, on the basis of a total amount of the aqueous ophthalmic composition.

In addition, the content proportion of the cellulose-based polymeric compound to the content of zinc chloride is such that, for example, a total amount of the cellulose-based polymeric compounds is preferably from 0.002 to 100,000 parts by weight, more preferably from 0.5 to 60,000 parts by weight, and even more preferably from 2 to 30,000 parts by weight, based on 1 part by weight of zinc chloride.

(6) Other Components

The aqueous ophthalmic composition can selectively contain various pharmacologically active components and physiologically active components according to the conventional methods, depending upon the applications and formulation forms thereof, within the range that would not impair the effects of the present invention.

Further, the aqueous ophthalmic composition of the present invention can contain various additives according to the conventional methods, depending upon the applications and formulation forms thereof, within the range that would not impair the effects of the present invention.

As the other components, boric acid and/or a salt thereof, for example, borax and the like, and sodium chloride are especially preferable.

(7) Method of Preparation and Applications of Aqueous Ophthalmic Composition

The aqueous ophthalmic composition of the present invention may be any aqueous composition having a pH of 7 or more, containing a terpenoid and zinc chloride, without particular limitations, and the aqueous composition can be prepared according to a method known to one of ordinary skill in the art. For example, the aqueous composition can be produced by dissolving each of the components in a proper amount of purified water, thereafter adjusting to a given pH value, and subsequently adding the remaining purified water to adjust its volume. In addition, the aqueous composition may also be optionally subjected to filtration and sterilization treatment, and then filled to the container.

Therefore, from another aspect, the present invention provides a method for producing an aqueous ophthalmic composition having an action of inhibiting adsorption of the terpenoid to a container, an enhanced inhibitory action for histamine release, and an inhibitory action for discharges from the eyes, including adding a terpenoid and zinc chloride to a carrier containing water, to provide an aqueous composition having a pH of 7 or more.

The aqueous ophthalmic composition of the present invention can be used as formulations such as medicaments and quasi-drugs, including eye drops [the eye drops including eye drops which can be instilled into the eyes while wearing contact lenses], artificial tears, eyewashes [the eyewashes including eyewashes which can wash the eyes while wearing contact lenses], compositions for contact lenses [solutions for wearing contact lenses, compositions for contact lenses care (disinfectant solutions for contact lens, storage solutions for contact lens, cleansing solutions for contact lenses, cleansing-storage solutions for contact lenses), and the like], and the like. One preferred example of the aqueous ophthalmic composition of the present invention includes eye drops, artificial tears, eyewashes, and solutions for wearing contact lenses, and especially preferred example includes eye drops and artificial tears. Here, when used as compositions for contact lenses, the aqueous ophthalmic composition is applicable to all sorts of contact lenses, including hard contact lenses and soft contact lenses.

The aqueous ophthalmic composition of the present invention can be provided by housing the aqueous ophthalmic composition in any sorts of containers. The container for housing the aqueous ophthalmic composition of the present invention is not particularly limited, and any container made of materials that can be used in a general container in the field of the art may be used, and, for example, glass materials and plastic materials, e.g. polyethylene terephthalate resin, polypropylene resin, polyethylene resin, polyethylene naphthalate resin, and the like, may be properly selected and used according to their purposes and applications. In addition, the container for housing the aqueous ophthalmic composition of the present invention may be a transparent container in which the internal of the container can be visually recognized, or an opaque container which is difficult to visually recognize the internal. Since the confirmation of the amount of the solution of the aqueous ophthalmic composition, foreign objects tests during the production steps, and the like is facilitated, especially transparent containers are preferred. Here, the term “transparent container” includes both colorless transparent containers and colored transparent containers.

The aqueous ophthalmic composition of the present invention can especially remarkably inhibit the reduction in the content of the terpenoid by adsorption of the terpenoid to the container, even when the aqueous ophthalmic composition is housed in a plastic container which is more likely to cause adsorption of the terpenoid in the conventional aqueous ophthalmic composition. For this reason, the aqueous ophthalmic composition of the present invention is highly useful as an aqueous ophthalmic composition used after housing in a plastic container, and especially highly useful as an aqueous ophthalmic composition housed in a container made of a material including a polyethylene terephthalate resin or polyethylene resin to which terpenoid is more likely to be adsorbed.

Further, the aqueous ophthalmic composition of the present invention is provided not only as a package form of “fully use at once” type, but also is useful as an aqueous ophthalmic composition of multi-dose which is a package in the form that is administered over plural times, and the user continuously doses.

### 2. Method for Inhibiting Adsorption of Terpenoid in Aqueous Ophthalmic Composition to Container

As mentioned above, since in the aqueous ophthalmic composition zinc chloride and are combined together with a terpenoid, the terpenoid contained in the aqueous ophthalmic composition having a pH of 7 or more can be inhibited from being adsorbed to a container, such as a plastic container, especially a container made of polyethylene terephthalate, thereby inhibiting the reduction in the terpenoid content in the aqueous ophthalmic composition.

Therefore, from another aspect, the present invention provides a method for inhibiting adsorption of the terpenoid in the aqueous ophthalmic composition having a pH of 7 or more to a container, or a method for inhibiting the reduction in the content of the terpenoid, including combining in the aqueous ophthalmic composition of the present invention zinc chloride together with a terpenoid.

Further, from another aspect, the present invention provides use of a terpenoid and zinc chloride, in the manufacture of an aqueous ophthalmic composition having a pH of 7 or more, containing a terpenoid and zinc chloride, the aqueous composition having an action for inhibiting adsorption of the terpenoid to a container.

Further, from another aspect, the present invention provides use of a composition having a pH of 7 or more, containing a terpenoid and zinc chloride, as an aqueous ophthalmic composition having an action for inhibiting adsorption of the terpenoid to a container.

Further, from another aspect, the present invention provides an aqueous composition having a pH of 7 or more, containing a terpenoid and zinc chloride, for use in an aqueous ophthalmic composition having an action for inhibiting adsorption of the terpenoid to a container.

In these methods, use, and compositions, so long as the terpenoid and zinc chloride are co-present in an aqueous ophthalmic composition, and the order of addition of those components are not particularly limited. The terpenoid and zinc chloride may be those that can be combined in the aqueous ophthalmic composition of the present invention, and amounts thereof may be amount that can be combined in the aqueous ophthalmic composition of the present invention. Also, the kinds and contents of each of the components combined in the aqueous ophthalmic composition, the kinds and contents of the other components combined, formulation forms of the composition, and the like are the same as the aqueous ophthalmic composition of the present invention.

### 3. Method for Enhancing Inhibitory Action for Histamine Release and Method for Inhibiting or Treating Itchiness of the Eyes

Further, as mentioned above, by providing an aqueous ophthalmic composition having a pH of 7 or more, containing a terpenoid and zinc chloride, inhibitory action for histamine release in the aqueous ophthalmic composition can be enhanced.

Accordingly, from another aspect, the present invention provides a method for enhancing inhibitory action for histamine release of an aqueous ophthalmic composition having a pH of 7 or more, including combining in the aqueous ophthalmic composition a terpenoid and zinc chloride.

Further, from another aspect, the present invention provides use of a terpenoid and zinc chloride, in the manufacture of an aqueous ophthalmic composition having a pH of 7 or more, containing a terpenoid and zinc chloride, the aqueous composition having an enhanced inhibitory action for histamine release.

Further, from another aspect, the present invention provides use of an aqueous composition having a pH of 7 or more, containing a terpenoid and zinc chloride, as an aqueous ophthalmic composition having an enhanced inhibitory action for histamine release.

Further, from another aspect, the present invention provides an aqueous composition having a pH of 7 or more, containing a terpenoid and zinc chloride, for use in an aqueous ophthalmic composition having an enhanced inhibitory action for histamine release.

In addition, as mentioned above, by contacting the aqueous ophthalmic composition of the present invention with the cornea and/or conjunctiva by a method such as eye dropping or washing eyes using the composition as eye drops or eyewashes, the action for inhibiting histamine release is enhanced, and whereby consequently anti-histamine action is enhanced, so that itchiness of the eyes can be inhibited or treated.

Therefore, further, from another aspect, the present invention provides a method for inhibiting or treating itchiness of the eyes, including contacting an aqueous ophthalmic composition having a pH of 7 or more, containing a terpenoid and zinc chloride, with the cornea and/or conjunctiva.

In these methods, use, and compositions, so long as a terpenoid and zinc chloride are co-present in the aqueous ophthalmic composition, and the order of addition of those components are not particularly limited. A terpenoid and zinc chloride may be those that can be combined in the aqueous ophthalmic composition of the present invention, and amounts thereof may be amounts that can be combined in the aqueous ophthalmic composition of the present invention. Also, the kinds and contents of each of the components to be combined in the aqueous ophthalmic composition, the kinds and contents of the other components to be combined, formulation forms of the composition, and the like are the same as the aqueous ophthalmic composition of the present invention.

### 4. Method for Inhibiting Discharges from the Eyes

Further, as mentioned above, by providing an aqueous ophthalmic composition having a pH of 7 or more, containing zinc chloride together with a terpenoid, an action for inhibiting discharges from the eyes can be given to the aqueous ophthalmic composition.

Accordingly, from another aspect, the present invention provides a method for providing an action for inhibiting discharges from the eyes to an aqueous ophthalmic composition having a pH of 7 or more, including combining in the aqueous ophthalmic composition a terpenoid and zinc chloride.

Further, from another aspect, the present invention provides use of a terpenoid and zinc chloride, in the manufacture of an aqueous ophthalmic composition having a pH of 7 or more, containing a terpenoid and zinc chloride, the aqueous composition having an inhibitory action for discharges from the eyes.

Further, from another aspect, the present invention provides use of a composition having a pH of 7 or more, containing a terpenoid and zinc chloride, as an aqueous ophthalmic composition having an inhibitory action for discharges from the eyes.

Further, from another aspect, the present invention provides an aqueous composition having a pH of 7 or more, containing a terpenoid and zinc chloride, for use in an aqueous ophthalmic composition having an inhibitory action for discharges from the eyes.

In addition, as mentioned above, by contacting an aqueous ophthalmic composition of the present invention with the cornea and/or conjunctiva by a method such as eye dropping or washing eyes using the composition as eye drops or eyewashes, the discharges from the eyes can be inhibited, so that ease in opening eyes, ease in blinking, blurriness of eyes, appreciative appearance of eyes or the like can be ameliorated.

Therefore, further, from another aspect, the present invention provides a method for inhibiting discharges from the eyes, including contacting an aqueous ophthalmic composition having a pH of 7 or more, containing a terpenoid and zinc chloride, with the cornea and/or conjunctiva.

In these methods, use, and compositions, so long as a terpenoid and zinc chloride are co-present in the aqueous ophthalmic composition, the order of addition of those components is not particularly limited. A terpenoid and zinc chloride may be those that can be combined in the aqueous ophthalmic composition of the present invention, and amounts thereof may be amount that can be combined in the aqueous ophthalmic composition of the present invention. Also, the kinds and contents of each of the components to be combined in the aqueous ophthalmic composition, the kinds and contents of the other components to be combined, formulation forms of the composition, and the like are the same as the aqueous ophthalmic composition of the present invention.

### EXAMPLES

The present invention will be described in detail hereinbelow by means of Examples and Test Examples, without intending to limit the present invention to these Examples and the like.

### Test Example 1

### Inhibition for Adsorption of Terpenoid

Each of the aqueous ophthalmic compositions of Comparative Examples 1 to 7 and Examples 1 and 2 having a composition as listed in Table 1 was prepared, and 8 mL each was dispensed in a 10 mL eye drop container made of polyethylene terephthalate (hereinafter also referred to as “PET”). Thereafter, a nozzle made of polyethylene (hereinafter also referred to as “PE”) and a cap were fitted thereto. The units for the content proportions of the components in Table 1 are w/v %.

Next, these containers were allowed to stand in a thermostat held at 60° C. for one week. Here, “allowed (allowing) to stand” refers to a state of allowing to stand without applying shaking. The content of the l-menthol in the test solution immediately after the preparation and the content after allowing the test solution to stand at 60° C. for one week were measured according to a conventional method using gas chromatography.

The residual rate of l-menthol in the test solution (formula (1)) was calculated in accordance with the following formula (1).

$$Residual Rate ( \% ) = \frac{\begin{matrix}1 \text{-} Menthol Content After Allowing \\ to Stand at 60 ° C . for One Week\end{matrix}}{\begin{matrix}1 \text{-} Menthol Content \\ Immediately After the Preparation\end{matrix}} \times 100 \qquad formula ( 1 )$$

Also, as to each of Examples 1 and 2, and Comparative Examples 2, 3, 5 and 7, an aqueous ophthalmic composition which does not contain a zinc compound, but a composition of all other components and pH are identical was used as a corresponding Comparative Example, and the rate of inhibiting adsorption of l-menthol to the container was calculated in accordance with the following formulas (2) and (3). Concretely, as the corresponding Comparative Example, one that corresponds to Comparative Examples 2 and 3 is Comparative Example 1, one that corresponds to Example 1 and Comparative Example 5 is Comparative Example 4, and one that corresponds to Example 2 and Comparative Example 7 is Comparative Example 6.

$$\begin{matrix}Rate of Adsorption of 1 \text{-} Menthol to the Container ( \% ) = 100 ( \% ) - Residual Rate ( \% ) & formula ( 2 ) \\ Rate of Inhibiting Adsorption of 1 \text{-} Menthol ( \% ) = \frac{\begin{matrix}Rate of Adsorption ( \% ) of Corresponding \\ Comparative Example - Rate of Adsorption ( \% )\end{matrix}}{\begin{matrix}Rate of Adsorption ( \% ) of Corresponding \\ Comparative Example\end{matrix}} \times 100 & formula ( 3 )\end{matrix}$$

The results are shown in the following Table 1.

TABLE 1

| | Comp. | Comp. | Comp. | Comp. | | Comp. | Comp. | | Comp. |
|---|---|---|---|---|---|---|---|---|---|
| Test Solution Name | Ex. 1 | Ex. 2 | Ex. 3 | Ex. 4 | Ex. 1 | Ex. 5 | Ex. 6 | Ex. 2 | Ex. 7 |
| Zinc Chloride | — | 0.01 | — | — | 0.01 | — | — | 0.01 | — |
| Zinc Sulfate | — | — | 0.01 | — | — | 0.01 | — | — | 0.01 |
| l-Menthol | 0.015 | 0.015 | 0.015 | 0.015 | 0.015 | 0.015 | 0.015 | 0.015 | 0.015 |
| Polysorbate 80 | 0.05 | 0.05 | 0.05 | 0.05 | 0.05 | 0.05 | 0.05 | 0.05 | 0.05 |
| Hydrochloric Acid | q.s. | q.s. | q.s. | q.s. | q.s. | q.s. | q.s. | q.s. | q.s. |
| Sodium Hydroxide | q.s. | q.s. | q.s. | q.s. | q.s. | q.s. | q.s. | q.s. | q.s. |
| Purified Water | q.s. | q.s. | q.s. | q.s. | q.s. | q.s. | q.s. | q.s. | q.s. |
| pH (r.t.) | 6.5 | 6.5 | 6.5 | 7 | 7 | 7 | 8 | 8 | 8 |
| Rate of Adsorption | 29.5 | 29.3 | 30.2 | 32.3 | 29.1 | 32.1 | 41.3 | 34.8 | 43.0 |
| of l-Menthol to | | | | | | | | | |
| Container (%) | | | | | | | | | |
| Rate of Inhibiting | — | 0.7 | −2.4 | — | 9.91 | 0.6 | — | 15.7 | −4.1 |
| Adsorption of | | | | | | | | | |
| l-Menthol (%) | | | | | | | | | |

As is clear from the results of Comparative Examples 1 to 3 in Table 1, as to the aqueous ophthalmic composition having a pH of 6.5, when the compositions containing zinc chloride or zinc sulfate and compositions not containing them are compared, the aqueous ophthalmic compositions were found to have hardly any differences in the rate of adsorption of l-menthol, and were not found to have a great difference in the rate of inhibiting adsorption of l-menthol by combining the zinc compound, so that the adsorption of l-menthol to a container was not inhibited by combining the zinc compound.

On the other hand, as is clear from the results of Comparative Examples 4 and 5 and Example 1, in the aqueous ophthalmic compositions having a pH of 7, the aqueous ophthalmic compositions were found to have hardly any differences in the rate of adsorption of l-menthol between the composition not containing both of zinc chloride and zinc sulfate and the composition containing zinc sulfate. However, the composition containing zinc chloride was found to have a decreased rate of adsorption of l-menthol, and a greatly improved rate of inhibiting adsorption of l-menthol, so that that the inhibition of adsorption of l-menthol to a container could be confirmed.

Also, from the results of Comparative Examples 6 and 7 and Example 2, similar results were found for the aqueous ophthalmic composition having a pH of 8.

In addition, the higher the pH, the higher the rate of adsorption of l-menthol to a container, so that the disadvantages were clarified to be greater.

Accordingly, it could be confirmed that by combining zinc chloride in an aqueous ophthalmic composition having a pH of 7 or more, the adsorption of l-menthol to a container can be inhibited.

### Test Example 2

### Inhibition 2 for Adsorption of Terpenoid

Test solutions were prepared in accordance with a formulation as listed in Tables 2 and 3, the content of a terpenoid (1-menthol or d-borneol) was measured in the same manner as in Test Example 1, and the residual rate of the terpenoid in the test solution was calculated in accordance with the formula (1), provided that in Comparative Example 12 and Example 9, a container made of polyethylene was used as an eye drop container in place of a container made of PET. In addition, the rate of inhibiting adsorption of the terpenoid to a container was calculated in accordance with the formulas (2) and (3). The results are also shown together in Tables 2 and 3. The units for the content proportions of the components in Tables 2 and 3 are w/v %.

The phrase “corresponding Comparative Examples” as used herein concretely refers to an aqueous ophthalmic composition which does not contain a zinc compound, but a composition of all other components and pH are identical, or refers to an aqueous ophthalmic composition which does not contain zinc chloride and a polymeric compound, but a composition of all other components and pH are identical, and the correspondences are concretely as in the following Table 4.

TABLE 2

| | Comp. | Comp. | Comp. | Comp. | Comp. |
|---|---|---|---|---|---|
| Test Solution Name | Ex. 8 | Ex. 9 | Ex. 10 | Ex. 11 | Ex. 12 |
| l-Menthol | 0.015 | 0.005 | — | 0.01 | 0.015 |
| d-Borneol | — | — | 0.005 | — | — |
| Polysorbate 80 | 0.05 | 0.05 | 0.05 | 0.04 | 0.05 |
| Hydroxyethyl | — | — | 1 | 0.01 | — |
| Cellulose | | | | | |
| Hydrochloric Acid | q.s. | q.s. | q.s. | q.s. | q.s. |
| Sodium Hydroxide | q.s. | q.s. | q.s. | q.s. | q.s. |
| Purified Water | bal. | bal. | bal. | bal. | bal. |
| pH (r.t.) | 7.8 | 7.8 | 7.8 | 7.8 | 7.8 |

TABLE 3

| Test Solution Name | Ex. 3 | Ex. 4 | Ex. 5 | Ex. 6 | Ex. 7 | Ex. 8 | Ex. 9 |
|---|---|---|---|---|---|---|---|
| Zinc Chloride | 0.003 | 0.0001 | 0.003 | 0.0001 | 0.0005 | 0.008 | 0.003 |
| l-Menthol | 0.015 | 0.005 | 0.015 | 0.015 | — | 0.01 | 0.015 |
| d-Borneol | — | — | — | — | 0.005 | — | — |
| Polysorbate 80 | 0.05 | 0.05 | 0.05 | 0.05 | 0.05 | 0.04 | 0.05 |
| Hypromellose | — | — | — | 0.6 | — | — | — |
| Hydroxyethyl | — | — | 1 | — | 1 | 0.01 | — |
| Cellulose | | | | | | | |
| Hydrochloric Acid | q.s. | q.s. | q.s. | q.s. | q.s. | q.s. | q.s. |
| Sodium Hydroxide | q.s. | q.s. | q.s. | q.s. | q.s. | q.s. | q.s. |
| Purified Water | bal. | bal. | bal. | bal. | bal. | bal. | bal. |
| pH | 7.8 | 7.8 | 7.8 | 7.8 | 7.8 | 7.8 | 7.8 |
| Rate of Inhibiting | 4.1 | 3.1 | 8.9 | 20.8 | 12.7 | 3.1 | 2.0 |
| Adsorption of | | | | | | | |
| Terpenoid (%) | | | | | | | |

TABLE 4

| | Corresponding |
|---|---|
| Example | Comparative Examples |
| Examples 3, 5, 6 | Comparative Example 8 |
| Example 4 | Comparative Example 9 |
| Example 7 | Comparative Example 10 |
| Example 8 | Comparative Example 11 |
| Example 9 | Comparative Example 12 |

As is clear from Tables 2 and 3, the test solutions of Examples 3 to 9 containing zinc chloride and a terpenoid showed greatly improved rates of inhibiting adsorption of a terpenoid, as compared to the test solutions of corresponding Comparative Examples containing a terpenoid without containing zinc chloride, so that it could be confirmed that adsorption of a terpenoid to a container was inhibited by combining zinc chloride. In addition, the test solutions of Examples 5, 6 and 7 further containing a polymeric compound in addition to zinc chloride and a terpenoid showed even higher rates of inhibiting adsorption of a terpenoid. Also, as to a case of Example 9 using a container made of polyethylene, an effect of inhibiting adsorption of a terpenoid to a container was found in the same manner as the case using a PET container.

### Test Example 3

### Inhibition for Histamine Release

Rat basophil leukemia cell line (RBL-2H3) suspended in a DMEM medium (manufactured by Invitrogen) supplemented with a 10% by volume fetal bovine serum (manufactured by Invitrogen) was seeded to a 96-well microtiter plate (manufactured by Corning) at a density of 1.4×10⁵ cells/cm², and cultured at 37° C. under 5% CO₂ for 24 hours. Thereafter, the culture supernatant was removed by suction, a test solution as listed in Table 5 was added thereto in a volume of 0.1 ml each per well, and the cultured cells were incubated at 37° C. for 1 hour under 5% CO₂. Thereafter, the culture supernatant was removed by suction, PIPES buffer supplemented with a reagent A23187 (manufactured by SIGMA) so as to have a concentration of 10 μM was added thereto in a volume of 0.2 ml each per well, and the cultured cells were incubated at 37° C. for 30 minutes under 5% CO₂. Here, as the PIPES buffer, the following was used: pH 7.2, composition: 0.1 w/v % bovine serum albumin (manufactured by SIGMA), CaCl₂.2H₂O 3.0 mM, MgCl₂.6H₂O 0.40 mM, KCl 7.38 mM, NaCl 118.93 mM, D(+)-Glucose 5.60 mM, 25 mM PIPES (Piperazine-1,4-bis(2-ethanesulfonic acid)), manufactured by DOJINDO LABORATORIES.

The supernatant of each well was collected, and the concentration of histamine was quantified using an ELISA kit (manufactured by Oxford Biochemical Research).

Also, as a control, a test was conducted in the same manner as the method mentioned above to quantify a histamine concentration, except that the PIPES buffer was added to carry out incubation in place of the procedures in which a test solution was added to carry out incubation.

Further, as blank, a test was conducted in the same manner as the control to quantify a histamine concentration, except that PIPES buffer not supplemented with A23187 was added to carry out incubation in place of the procedures in which PIPES buffer supplemented with A23187 was added to carry out incubation.

Values resultant from subtracting the histamine concentration of the blank from the histamine concentrations of each sample and the control are defined as a true histamine concentration of each sample and a true histamine concentration of the control, respectively. Using the true histamine concentration of each sample and the true histamine concentration of the control obtained, the rate of inhibiting histamine release (%) was calculated in accordance with the following formula (4).

$$Rate of Inhibiting Histamine Release ( \% ) = \{ 1 - \frac{\begin{matrix}True Histamine Concentration of \\ Each Test Solution\end{matrix}}{True Histamine Concentration of Control} \} \times 100 \qquad ( 4 )$$

Further, based on the rate of inhibiting histamine release calculated according to the above method, as to Comparative Examples 14 to 16 using a test solution having a pH of 6.5, an increased amount of rate of inhibiting histamine release based on Comparative Example 13 was calculated in accordance with the following formula (5), on the basis of the rate of inhibiting histamine release in Comparative Example 13 using a test solution having a pH of 6.5, not containing both of zinc chloride and 1-menthol. As to Comparative Examples 18 and 19 and Example 10 using a test solution having a pH of 7.0, an increased amount of rate of inhibiting histamine release based on Comparative Example 17 was calculated in accordance with the following formula (5), on the basis of the rate of inhibiting histamine release in Comparative Example 17 using a test solution having a pH of 7.0, not containing both of zinc chloride and 1-menthol. The results are together shown in Table 5.

$$Increased Amount of Rate of Inhibiting Histamine Release ( \% ) = Rate of Inhibiting Histamine Release in Comparative Example or Example - Rate of Inhibiting Histamine Release in Comparative Example 13 or Comparative Example 17 \qquad ( 5 )$$

Here, as the standards of the components used in the test, zinc chloride is a reagent manufactured by Wako Pure Chemical Industries, Ltd., and l-menthol is a reagent manufactured by Wako Pure Chemical Industries, Ltd. Also, units of the content proportions of the components in Table 5 are w/v %.

TABLE 5

| | Comp. | Comp. | Comp. | Comp. | Comp. | Comp. | Comp. | |
|---|---|---|---|---|---|---|---|---|
| | Ex. 13 | Ex. 14 | Ex. 15 | Ex. 16 | Ex. 17 | Ex. 18 | Ex. 19 | Ex. 10 |
| Zinc Chloride | — | — | 0.001 | 0.001 | — | — | 0.001 | 0.001 |
| l-Menthol | — | 0.01 | — | 0.01 | — | 0.01 | — | 0.01 |
| Polysorbate 80 | 0.03 | 0.03 | 0.03 | 0.03 | 0.03 | 0.03 | 0.03 | 0.03 |
| Boric acid | 0.2 | 0.2 | 0.2 | 0.2 | 0.2 | 0.2 | 0.2 | 0.2 |
| Borax | q.s. | q.s. | q.s. | q.s. | q.s. | q.s. | q.s. | q.s. |
| Sodium Chloride | 0.8 | 0.8 | 0.8 | 0.8 | 0.8 | 0.8 | 0.8 | 0.8 |
| Purified Water | q.s. | q.s. | q.s. | q.s. | q.s. | q.s. | q.s. | q.s. |
| pH | 6.5 | 6.5 | 6.5 | 6.5 | 7.0 | 7.0 | 7.0 | 7.0 |
| Increased Amount of | — | −16.7 | −4.4 | −12.5 | — | — | — | — |
| Rate of Inhibiting | | | | | | | | |
| Histamine Release | | | | | | | | |
| Based on Comp. Ex. | | | | | | | | |
| 13 (%) | | | | | | | | |
| Increased Amount of | — | — | — | — | — | −9.8 | 4.5 | 10.6 |
| Rate of Inhibiting | | | | | | | | |
| Histamine Release | | | | | | | | |
| Based on Comp. Ex. | | | | | | | | |
| 17 (%) | | | | | | | | |

As is clear from Table 5, Comparative Examples 14 and 18 using a test solution in which menthol was combined in the test solution of Comparative Example 13 or 17 were both likely to have lowered inhibitory effects for histamine release, as compared to that of Comparative Example 13 or 17. Further, Comparative Example 16 using a test solution having a pH of 6.5 in which menthol and zinc chloride were combined in the test solution of Comparative Example 13 was hardly found to have any improvements in inhibitory effects for histamine release.

By contrast, Example 10 using a test solution having a pH of 7.0 in which menthol and zinc chloride were combined in the test solution of Comparative Example 17 had remarkable improvements in the effects of inhibiting histamine release, and exhibited high effects of inhibiting histamine release.

### Test Example 4

### Inhibitory Test for Discharges from the Eyes

Test solutions were prepared in accordance with a formulation as listed in Table 6 and 7, and filled in an eye drop container made of PET to give a test sample. A tested individual was instilled into the eyes two kinds of test samples as one set, one set of the test sample instilling into the right eye, and the other set instilling into the left eye, one set per day, five times a day, 2 drops each time (n=10). Here, one kind of the test sample was always instilled into the identical eye, and the intervals of instillations between each time were kept one or more hours. After 2 hours or more from the fifth instillation into the eyes, the amount of discharges from the eyes which the tested individual subjectively sensed was evaluated in accordance with a visual analogue scale method (VAS method). In other words, on a straight line of 10 cm in length, the left end of the straight line, i.e. a point at 0 cm is defined as “no discharges from the eyes being found,” and the right end of the straight line, i.e. a point at 10 cm is defined as “maximum amount of discharges from the eyes which was experienced in the past being found,” the tested individual was asked to show one point on the straight line that corresponds to the amount of discharges from the eyes which is subjectively sensed in the eyes after the instillation into the eyes of each of test samples, the distance (cm) from the point at 0 cm was measured and defined as scores on the amount of discharges from the eyes. The results are together shown in Tables 6 and 7. The units of the content proportions of the components in Tables 6 and 7 are w/v %.

TABLE 6

| | Name of Set | | | | | | | |
|---|---|---|---|---|---|---|---|---|
| | Set A | | Set B | | Set C | | Set D | |
| | Right Eye/Left Eye | | | | | | | |
| | Right | Left | Right | Left | Right | Left | Right | Left |
| | Eye | Eye | Eye | Eye | Eye | Eye | Eye | Eye |
| | Test Solution Name | | | | | | | |
| | Comp. | Comp. | Comp. | | Comp. | | Comp. | |
| | Ex. 20 | Ex. 21 | Ex. 22 | Ex. 11 | Ex. 23 | Ex. 12 | Ex. 24 | Ex. 13 |
| Zinc Chloride | — | 0.002 | — | 0.002 | — | 0.002 | — | 0.002 |
| l-Menthol | — | — | 0.05 | 0.05 | 0.01 | 0.01 | 0.01 | 0.01 |
| Polysorbate 80 | 0.04 | 0.04 | 0.2 | 0.2 | 0.04 | 0.04 | 0.04 | 0.04 |
| Chlorobutanol | | | 0.35 | 0.35 | | | | |
| Hydroxyethyl | — | — | — | — | — | — | 0.01 | 0.01 |
| Cellulose | | | | | | | | |
| Boric Acid | 1 | 1 | 1 | 1 | 1 | 1 | 1 | 1 |
| Sodium Chloride | 0.4 | 0.4 | 0.4 | 0.4 | 0.4 | 0.4 | 0.4 | 0.4 |
| Borax | q.s. | q.s. | q.s. | q.s. | q.s. | q.s. | q.s. | q.s. |
| Purified Water | q.s. | q.s. | q.s. | q.s. | q.s. | q.s. | q.s. | q.s. |
| pH | 7.2 | 7.2 | 7.2 | 7.2 | 7.2 | 7.2 | 7.2 | 7.2 |
| Amount of | 6.4 | 5.8 | 4.9 | 3.4 | 5.5 | 3.5 | 6.7 | 2.5 |
| Discharges from | | | | | | | | |
| the Eyes (Average) | | | | | | | | |

TABLE 7

| | |
|---|---|
| Name of Set | Set E |

| Right Eye/Left Eye | Right Eye | Left Eye |
|---|---|---|
| Test Solution Name | Comp. Ex. 20 | Comp. Ex. 25 |
| Zinc Chloride | — | 0.002 |
| l-Menthol | — | 0.01 |
| Polysorbate 80 | 0.04 | 0.04 |
| Boric Acid | 1 | 1 |
| Sodium Chloride | 0.4 | 0.4 |
| Borax | q.s. | q.s. |
| Purified Water | q.s. | q.s. |
| pH | 7.2 | 6.0 |
| Amount of Discharges | 6.4 | 7.0 |
| from the Eyes (Average) | | |

As is clear from Tables 6 and 7, the test solutions having a pH of 7.2, containing zinc chloride and menthol of Examples 11 to 13 were found to have remarkable inhibitory effects of the discharges from the eyes. Particularly, as is clear from comparison of Comparative 21 and 23 with Example 12, test solutions containing either one of zinc chloride or menthol of Comparative Examples 21 and 23 were found to have a slightly inhibitory effect of discharges from the eyes. By contrast, a test solution concomitantly containing both zinc chloride and menthol of Example 12 was found to have remarkable inhibitory effects of discharges from the eyes.

Incidentally, a test solution having a pH of 6.0 of Comparative Example 25 was not found to have inhibitory effects of discharges from the eyes, even when containing zinc chloride and menthol.

1. An aqueous ophthalmic composition having a pH of 7 or more, comprising a terpenoid and zinc chloride.

2. The aqueous ophthalmic composition according to claim 1, wherein the terpenoid is at least one member selected from the group consisting of menthol, menthone, camphor, borneol, and geraniol.

3. The aqueous ophthalmic composition according to claim 1 or 2, wherein the composition is housed in a container made of a material comprising at least one plastic selected from the group consisting of polyethylene terephthalate resins, polypropylene resins, polyethylene resins, and polyethylene naphthalate resins.

4. A method for inhibiting adsorption of a terpenoid in an aqueous ophthalmic composition having a pH of 7 or more to a container, comprising combining in the aqueous ophthalmic composition zinc chloride together with a terpenoid.

5. A method for enhancing an inhibitory action for histamine release of an aqueous ophthalmic composition having a pH of 7 or more, comprising combining in the aqueous ophthalmic composition zinc chloride together with a terpenoid.

6. A method for giving an inhibitory action for discharges from the eyes to an aqueous ophthalmic composition having a pH of 7 or more, comprising combining in the aqueous ophthalmic composition zinc chloride together with a terpenoid.
